(19) United States
(12) Patent Application Publication
(10) Pub. No.: US 2012/0245382 A1
(43) Pub. Date: Sep. 27, 2012

(54) PRODUCING ACETALDEHYDE AND/OR ACETIC ACID FROM BIOETHANOL

(75) Inventors: Sabine Huber, Limburgerhof (DE); Markus Gitter, Brussels (BE); Ulrich Cremer, Ludwigshafen (DE)

(73) Assignee: BASF SE, Ludwigshafen (DE)

(21) Appl. No.: 13/513,755

(22) PCT Filed: Dec. 3, 2010
(86) PCT No.: PCT/EP10/68793

§ 371 Date: Jun. 4, 2012

(30) Foreign Application Priority Data
Dec. 4, 2009 (EP) 09178015.5

(51) Int. Cl. C07C 51/235

(52) U.S. Cl. 562/535; 562/532

(57) ABSTRACT

The invention discloses a method for producing acetaldehyde and/or acetic acid, according to which method a gaseous flow, containing molecular oxygen, ethanol and at least one impurity selected from sulphur compounds, is brought into contact at a high temperature with a sulphur-resistant oxidation catalyst. The ethanol is preferably obtained from a biomass. Said sulphur-resistant oxidation catalyst comprises, for example, vanadium oxide and at least one oxide of zirconium, titanium and aluminium. In one embodiment, the gaseous flow is converted, on the sulphur-resistant oxidation catalyst, into a first oxidation mixture, acetaldehyde being the predominant oxidation product, and said first oxidation mixture is converted, on another oxidation catalyst, into a second oxidation mixture, acetic acid being the predominant oxidation product. Said other oxidation catalyst comprises, for example, a multi-metal oxide containing at least molybdenum and vanadium.

The present invention relates to a process for preparing acetaldehyde and/or acetic acid from ethanol which comprises at least one impurity selected from sulfur compounds, especially from bioethanol.

The preparation of acetic acid by oxidation of ethanol is known. On the industrial scale, heterogeneously catalyzed reactions in particular are performed in the gas phase, since this does not require a removal of the catalyst from the oxidation product.

GB 1 301 145 describes a process for preparing an aliphatic monocarboxylic acid from an alkanol having two to four carbon atoms, in which the alkanol is introduced in vapor form into a reaction zone containing a solid catalyst comprising palladium metal and reacted with an oxygenous gas.

EP-A 0294846 describes a process for preparing an organic acid by catalytic oxidation of an alcohol in contact with a calcined mixed oxide catalyst of the composition: MoₓV_{y}Z_{z} in which Z is absent or is a particular metal.

U.S. Pat. No. 5,840,971 discloses a process for preparing acetic acid by controlled oxidation of ethanol. The reaction is effected in the presence of a catalyst, the active composition of which consists of vanadium, titanium and oxygen.

DE 1097969 describes a process for preparing aldehydes by dehydrogenating primary aliphatic alcohols using a copper catalyst activated with chromates.

An increasingly useful starting material for acetic acid preparation is bioethanol. Bioethanol refers to ethanol which has been produced exclusively from biomass, i.e. renewable carbon carriers. The polysaccharides present in the biomass, in the form of starch or cellulose, are split enzymatically to give glucose which is subsequently fermented to ethanol.

As a result of the production, bioethanol comprises impurities, especially sulfur compounds. Sulfur compounds are effective catalyst poisons which can lead to the formation of catalysis-inactive metal sulfides on many catalyst surfaces, especially of noble metals. Purification of the bioethanol to remove the sulfur compounds is inappropriate for economic reasons.

It is therefore an object of the invention to specify a process for preparing acetaldehyde and/or acetic acid from bioethanol, in which a preceding purification of the bioethanol is not required.

The object is achieved by a process for preparing acetaldehyde and/or acetic acid, wherein a gaseous stream which comprises molecular oxygen, ethanol and at least one impurity selected from sulfur compounds is contacted at elevated temperature with a sulfur-resistant oxidation catalyst.

The ethanol is preferably bioethanol, i.e. ethanol which has been obtained from biomass. The gaseous stream comprises generally 2 to 100 ppm, usually 5 to 50 ppm, of sulfur compounds, based on the ethanol content. The content of sulfur compounds can be determined by gas chromatography. The sulfur compounds comprise organic sulfur compounds, especially dirnethyl sulfate and/or dimethyl sulfoxide.

An oxidation catalyst is referred to as “sulfur-resistant” when the concentration of organic sulfur compounds, e.g. dimethyl sulfoxide, in the ethanol used, which is required to lower the activity of the catalyst to 90% (of the initial activity) within 200 operating hours, is greater than 500 ppm (based on the ethanol content). The activity can suitably be determined as the ethanol conversion at a catalyst hourly space velocity of 50-200 gₑₜₕₐₙₒₗ/l.h, e.g. at 80 g of ethanol/l cat. and hour.

Preferred sulfur-resistant oxidation catalysts comprise vanadium oxide as the catalytically active constituent; more preferred catalysts comprise, as well as vanadium oxide, at least one oxide of zirconium, titanium and/or aluminum.

Catalysts comprising vanadium oxide are known per se. They are obtainable for example, by the following processes:
i) impregnating a porous support with a solution of a vanadium compound, removing the solvent and calcining the impregnated support; see, for example, U.S. Pat. No. 4,048,112;
ii) treating finely divided titanium dioxide with a vanadium compound, optionally applying the composition to an inert support and calcining; see, for example, U.S. Pat. No. 3,464,930:
iii) treating titanium dioxide with water and vanadium oxychloride, until the desired vanadium content has been attained; see, for example, U.S. Pat. No. 4,228,038;
iv) neutralizing a hydrochloric acid solution of vanadium pentoxide and titanium tetrachloride; see, for example, U.S. Pat. No. 3,954,857;
v) mixing a vanadyl alkoxide with a titanium alkoxide in an aqueous solution and calcining the precipitate; see, for example, U.S. Pat. No. 4,448,897.

Owing to the readily-available starting materials, preparation method (i) is generally preferred. Suitable porous supports are, for example, zirconium dioxide, titanium dioxide or aluminum oxide. The support may assume any suitable form, for example spheres, rings, pellets, extrudates or honeycomb form. It may suitably have a mean particle size of 2.5 to 10 mm. Suitable vanadium compounds are, for example, vanadium pentoxide or a vanadium salt such as vanadyl sulfate, vanadyl chloride or ammonium metavanadate, which are preferably dissolved in water in the presence of a complexing agent, such as oxalic acid.

The impregnation may be followed by an optional drying step, in which the solvent is removed, for example, at a temperature of 100 to 200° C. The impregnated support is then calcined at a temperature of at least 450° C., e.g., 500 to 800° C. The calcining can be effected in the presence of oxygen, for example under air, or in an inert atmosphere.

The dried and/or calcined support can optionally be impregnated again in order to achieve a desired loading with vanadium oxide.

In preparation method (ii), finely divided titanium dioxide, preferably in the anatase polymorph, is treated with a vanadium compound, for example with a solution of a vanadium compound in water or an organic solvent, such as formamide, mono- or polyhydric alcohols. The solution may optionally comprise complexing agents, such as oxalic acid. Alternatively, the finely divided titanium dioxide can be treated under hydrothermal conditions with a sparingly soluble vanadium compound such as vanadium pentoxide.

The resulting composition can be used either in powder form or shaped to particular catalyst geometries, in which case the shaping may precede or follow the final calcination. For example, unsupported catalysts can be prepared from the powder form of the active material or the uncalcined precursor material thereof by compacting to the desired catalyst geometry (e.g. by tableting or extruding), in which case auxiliaries, for example, graphite or stearic acid as lubricant and/or shaping auxiliaries and reinforcing agents such as microfibers of glass, asbestos, silicon carbide or potassium titanate can optionally be added. Suitable unsupported catalyst geometries are, for example, solid cylinders or hollow cylinders with an external diameter and a length of 2 to 10 mm. the case of hollow cylinders, a wall thickness of 1 to 3 mm is appropriate.

Alternatively, the resulting pulverulent active material or the pulverulent precursor material thereof which is yet to be calcined is used to coat an inert support to obtain what is known as an eggshell catalyst. The coating of the support bodies to prepare the eggshell catalysts is generally performed in a suitable rotatable vessel, for example by spray application in a coating drum, coating in a fluidized bed or powder coating system.

Appropriately, a suspension of the material to be applied is used to coat the support bodies. The layer thickness of the material applied to the support body is appropriately selected within the range, for example, of 10 μm to 2 mm.

The support materials used may be customary catalyst supports, preference being given to nonporous supports. Suitable nonporous inert supports are materials which are essentially free of pores or have a low specific surface area, preferably less than 3 m²/g. Useful examples are quartz, fused silica, sintered silica, sintered or fused alumina, porcelain, sintered or fused silicates, such as aluminum silicate, magnesium silicate, zinc silicate, zirconium silicate and especially steatite. The support bodies may be of regular or irregular shape, preference being given to regularly shaped support bodies, for example spheres or hollow cylinders. It is suitable to use essentially nonporous supports composed of steatite. The support may suitably have a mean particle size of 1 to 10 mm.

The catalytically active compositions obtained according to preparation methods (iii) to (iv) can likewise be applied to an inert support as described above.

In general, the sulfur-resistant oxidation catalyst comprises 0.1 to 30% by weight, preferably 5 to 20% by weight, of V₂O₅, based on the total weight of the catalyst.

The reaction can be performed in any reactor for performing heterogeneously catalyzed reactions in the gas phase, and the catalyst may be arranged as a fixed bed or fluidized bed. Suitable examples are fluidized bed reactors, tube bundle reactors or microreactors. Tube bundle reactors and rnicroreactors are generally preferred.

A tube bundle reactor consists of a multitude of reactor tubes in which a fixed bed of the catalyst is arranged, which are surrounded by a heat carrier medium for heating and/or cooling. In general, the industrially used tube bundle reactors comprise more than three to several tens of thousands of reactor tubes connected in parallel.

Conventional reactors and microreactors differ by their characteristic dimensions and more particularly by the characteristic dimensions of the reaction zones thereof. The characteristic dimension of a unit, for example of a reactor, is understood in the context of the present invention to mean the smallest dimension at right angles to the flow direction. The characteristic dimension of the reaction zone of a microreactor is significantly less than that of a conventional reactor (for example at least by a factor of 10 or at least by a factor of 100 or at least by a factor of 1000) and is typically in the range from one hundred nanometers to a few millimeters. It is frequently in the range from 1 μm to 30 mm.

In general, the gaseous stream comprises 0.5 to 20% by volume, especially 1 to 5% by volume, of ethanol.

In general, the gaseous stream comprises 0.5 to 20% by volume, especially 5 to 10% by volume, of oxygen. in preferred embodiments, the gaseous stream also comprises water vapor, preferably in an amount of up to 40% by volume, for example 1 to 15% by volume. The presence of water vapor facilitates the desorption of the oxidation products from the catalyst surface and can also improve the removal of the heat of reaction.

The difference to 100% by volume generally consists of at least one inert gas, preferably nitrogen, for example atmospheric nitrogen.

The gaseous stream is generally converted over the oxidation catalyst at a temperature of 150 to 300° C., acetic acid being the predominant oxidation product at relatively high temperatures.

When acetic acid is the desired oxidation product, the conversion can be performed in one or more stages, especially in two stages. In the case of multistage performance, the intermediate oxidation mixture obtained after one stage is preferably not worked up but fed unchanged to the subsequent stage.

One possible embodiment of a two-stage process relates to a process wherein the gaseous stream is converted over the sulfur-resistant oxidation catalyst to a first oxidation mixture in which acetaldehyde is the predominant oxidation product, and the first oxidation mixture is converted over a further oxidation catalyst to a second oxidation mixture in which acetic acid is the predominant oxidation product.

The further oxidation catalyst may be arranged in the same reactor as a bed placed downstream of the bed of the sulfur-resistant oxidation catalyst. The term “downstream” relates to the flow direction of the gaseous stream. The reactor may have two temperature zones, in which case the temperature of the zone of the further oxidation catalyst can be controlled independently of the zone of the sulfur-resistant oxidation catalyst.

A suitable catalyst in the second stage is any gas phase oxidation catalyst which can oxidize aldehydes selectively to carboxylic acids. The oxidation catalyst preferably comprises a multimetal oxide which comprises at least molybdenum and vanadium. Such catalysts are used, for example, for the partial oxidation of acrolein to acrylic acid.

The two-stage oxidation of ethanol to acetic acid permits better control of the evolution of heat. The loading of the gas stream with ethanol can be increased. The oxidation of acetaldehyde to acetic acid over multimetal oxide active materials comprising Mo and V proceeds with high selectivity. A high acetic acid yield over both stages is achieved.

Such multimetal oxide active materials comprising Mo and V can be found, for example, in U.S. Pat. No. 3,775,474, U.S. Pat. No. 3,954,855, U.S. Pat. No. 3,893,951 and U.S. Pat. No. 4,339,355 or EP-A 614872 or EP-A 1041062 or WO 03/055835 or WO 03/057653. Also especially suitable are the multimetal oxide active materials of DE-A 10 32 5487, DE-A 10 325 488, EP-A 427508, DE-A 29 09 671, DE-C 31 51 805, DE-B 26 26 887, DE-A 43 02 991, EP-A 700 893, EP-A 714 700 and DE-A 19 73 6105. Particular preference is given in this context to the exemplary embodiments of EP-A 714 700 and of DE-A 19 73 6105.

Suitable multimetal oxide active materials correspond to the general formula I,

Mo₁₂VₐX¹_{b}X²_{c}X³_{d}X⁴ₑX⁵_{f}X⁶_{g}Oₙ (I)

in which the variables are each defined as follows:

X¹=W, Nb, Ta, Cr and/or Ce,

X²=Cu, Ni, Co, Fe, Mn and/or Zn,

X³=Sb and/or Si,

X⁴=one or more alkali metals,

X⁵=one or more alkaline earth metals,

X⁶=Si, Al, Ti and/or Zr,

a=1 to 6,

b=0.2 to 4,

c=0.5 to 1 8,

d=0 to 40,

e=0 to 2,

f=0 to 4,

g=0 to 40 and

n=a number which is determined by the valency and frequency of the elements other than oxygen in I.

In preferred embodiments, the variables are each defined as follows:

X¹=W, Nb, and/or Cr,

X²=Cu_{'} Ni, Co, and/or Fe,

X³=Sb,

X⁴=Na and/or K,

X⁵=Ca, Sr and/or Ba,

X⁶=Si, Al, and/or Ti,

a=1.5 to 5,

b=0.5 to 2,

c=0.5 to 3,

d=0 to 2,

e=0 to 0.2,

f=0 to 1 and

n=a number which is determined by the valency and frequency of the elements other than oxygen in I.

The multimetal oxide active materials comprising Mo and V, especially those of the general formula I, can be used either in powder form or shaped to particular catalyst geometries as unsupported catalysts. They can also be applied to preshaped inert catalysts supports.

The invention is illustrated in detail by the examples which follow,

### EXAMPLE 1

### Preparation of an Oxidation Catalyst Comprising Vanadium Oxide

380.0 g of water were initially charged in a 2 l beaker and heated to 55° C. During the heating, 220.0 g of oxalic acid dihydrate were added. After complete dissolution of the oxalic acid dihydrate, 116 g of V₂O₅ were added in small portions, in the course of which a deep blue vanadium complex formed. On completion of V₂O₅ addition, the solution was heated to 80° C., stirred for a further 10 min and then cooled to room temperature, 97.5 g of titanium dioxide powder (anatase polymorph, BET about 20 m²/g) were added to 135 ml of the solution thus obtained and dispersed with an Ultra-Turrax at 8000 rpm for about 3 min. The resulting dispersion was used to coat shaped support bodies. To this end, the dispersion was applied in a coating unit with the aid of a two-substance nozzle to 150 g of steatite span (diameter 1 to 1.5 mm) (internal temperature of the coating drum 120° C.; 200 rpm; atomization with about 250 l (STP)/h of compressed air). The coated support was transferred into a porcelain dish and calcined under air at 500° C. (heating ramp 3° C./min) in a muffle furnace for 3 h.

### EXAMPLE 2

### Ethanol Oxidation Over a Fixed Catalyst Bed

Ten ml of the catalyst from example 1 were installed as a fixed bed into an electrically heated vertical tubular reactor (diameter 15 mm, length 1000 mm). In the upper half of the bed toward the gas inlet, the catalyst was diluted with 75% by weight of steatite, and in the lower half with 66% by weight of steatite. The length of the catalyst bed was about 250 mm. At each side of the bed was arranged a layer of 300 mm of steatite spheres (diameter 2 to 3 mm). Below the steatite bed was a catalyst base of height about 100 mm.

The apparatus was heated externally to 240° C. Evaporated ethanol, evaporated water, air and nitrogen were supplied to the reactor. The composition of the gas stream was 1.4% by volume of ethanol, 14% by volume of H₂O, 5% by volume of O₂, remainder N₂. The ethanol used had a sulfur content of 3 ppm. The pressure in the reactor was 4 bar gauge. The hotspot temperature reached 260° C.

At a conversion of ethanol of more than 99%, a selectivity for acetaldehyde S_{acataldehyde} of 16 mol % and a selectivity for acetic acid S_{acetic acid} of 80.5 mol % were obtained.

### EXAMPLE 3

The experiment from example 2 was continued, except that ethanol to which 20 ppm (based on ethanol) of dimethyl sulfoxide had been added was used. The reaction was conducted under the same conditions for 400 h. No decline in the ethanol conversion was observed. The total selectivity S_{acetic acid}+S_{acetaldehyde} improved by 0.5 mol %.

### EXAMPLE 4

### Preparation of a Second-Stage Oxidation Catalyst

127 g of copper(II) acetate monohydrate were dissolved in 2700 g of water to give a solution I. 860 g of ammonium heptamolybdate tetrahydrate, 143 g of ammonium metavanadate and 126 g of ammonium paratungstate heptahydrate were dissolved successively at 95° C. in 5500 g of water to give a solution II. Subsequently, the solution I was stirred all at once into the solution II, and the aqueous mixture was spray-dried at an exit temperature of 110° C. Thereafter, the spray powder was kneaded with 0.15 kg of water per kg of powder. The kneaded mixture was calcined in a forced-air oven charged with an oxygen/nitrogen mixture. The oxygen content was adjusted such that there was an O₂ content of 1.5% by volume at the outlet of the forced-air oven. In the course of calcination, the kneaded material was heated first to 300° C. at a rate of 10 K/min and then held at this temperature over 6 h. Thereafter, it was heated to 400° C. at a rate of 10 K/min and this temperature was maintained for another 1 h. To adjust the ammonia content of the calcination atmosphere, the oven loading O (g of catalyst precursor per l of internal volume of the forced-air oven), the inlet volume flow IVF (l (STP)/h) of the oxygen/nitrogen mixture and the residence time RT (sec) of the oxygen/nitrogen charge (ratio of internal volume of the forced-air oven and volume flow of the oxygen/nitrogen mixture supplied) were selected as listed below. The forced-air oven used had an internal volume of 3 l. O: 250 g/l, RT: 135 sec and IVF: 80 l (STP)/h.

The resulting catalytically active material was based on the following stoichiometry Mo₁₂V₃W_{1.2}Cu_{1.6}Oₓ.

After grinding the calcined catalytically active material to particle diameters in the range from 0.1 to 50 μm, the resulting active material powder was used to coat, in a coating drum, nonporous steatite spheres having rough surfaces and a diameter of 2 to 3 mm with addition of water, so as to result in an active material content of 20% by weight. This was followed by drying with air at 110° C.

### EXAMPLE 5

### Two-Stage Ethanol Oxidation Over a Fixed Catalyst Bed

Ten ml of the catalyst from example 1 were diluted with 10 ml of steatite spall (1 to 1.5 mm) and installed as a fixed bed toward the gas inlet into an electrically heated tubular reactor (diameter 15 mm, length 1000 mm). Adjoining this first bed, 5 ml of the second-stage oxidation catalyst from example 4 were introduced.

The apparatus was heated externally to 185° C. in the region of the first bed, and to 220° C. in the region of the second-stage oxidation catalyst bed. Evaporated ethanol, evaporated water, air and nitrogen were supplied to the reactor. The composition of the gas stream was 1.6% by volume of ethanol, 10% by volume of H₂O, 6% by volume of O₂, remainder N₂.

At a conversion of ethanol of 99.8%, a selectivity for acetaldehyde S_{acetaldehyde} of 3 mol % and a selectivity for acetic acid S_{acetic acid} of 90 mol % were obtained.

1-11. (canceled)

12. A process for preparing acetic acid, wherein a gaseous stream which comprises molecular oxygen, ethanol and 2 to 100 ppm, based on the ethanol content, of at least one impurity selected from organic sulfur compounds is contacted at elevated temperature with a sulfur-resistant oxidation catalyst and the gaseous stream is converted over the sulfur-resistant oxidation catalyst to a first oxidation mixture in which acetaldehyde is the predominant oxidation product, and the first oxidation mixture is converted over a further oxidation catalyst to a second oxidation mixture in which acetic acid is the predominant oxidation product, wherein the sulfur-resistant oxidation catalyst comprises vanadium oxide, and the further oxidation catalyst comprises a multimetal oxide which comprises at least molybdenum and vanadium.

13. The process according, to claim 12, wherein the ethanol is obtained from biomass.

14. The process according to claim 12, wherein the sulfur-resistant oxidation catalyst, as well as vanadium oxide, comprises at least one oxide of zirconium, titanium and aluminum.

15. The process according to claim 12, wherein the sulfur-resistant oxidation catalyst is obtainable by impregnating a support with a solution of a vanadium compound and calcining the impregnated support.

16. The process according to claim 12, wherein the sulfur-resistant oxidation catalyst comprises 0.1 to 30% by weight of V2O5, based on the total weight of the catalyst.

17. The process according to claim 12, wherein the gaseous stream also comprises water vapor.

18. The process according to claim 12, wherein the gaseous stream is contacted with the sulfur-resistant oxidation catalyst at a temperature of 150 to 300° C.

19. The process according to claim 12, wherein the gaseous stream comprises 2 to 100 ppm of sulfur compounds.
